(19) United States
(12) Patent Application Publication
(10) Pub. No.: US 2017/0020708 A1
(43) Pub. Date: Jan. 26, 2017

(54) Abdominal and Thoracic Binder

(71) Applicant: Billy Forest Dye, Lakehills, TX (US)

(72) Inventor: Billy Forest Dye, Lakehills, TX (US)

(21) Appl. No.: 15/201,192

(22) Filed: Jul. 1, 2016

Related U.S. Application Data
(60) Provisional application No. 62/196,483, filed on Jul. 24, 2015.

(51) Int. Cl. A61F 5/03; A61F 5/02

(52) U.S. Cl. CPC A61F 5/03; A61F 5/02

(57) ABSTRACT

An abdominal and thoracic binder is used to provide support for back muscles and the abdomen of a user. The abdominal and thoracic binder includes a porous band, a fastener, a plurality of ventilation holes, and a plurality of reinforcing rods. The fastening layer is superimposed across and coextensive with the porous band. The porous band presses against the user's midsection and the fastening layer is exterior the porous band. The plurality of ventilation holes traverses through and is distributed across the porous band and the adhering layer, encouraging the breathability of the user's skin that is surrounded by the abdominal and thoracic binder. The plurality of reinforcing rods is detachably attached to the fastening layer and prevents the porous band and the fastening layer from folding. The abdominal and thoracic binder also includes a surface fastener that secures both the porous band and the fastening layer around the user.

The current application claims a priority to the U.S. Provisional Patent application Ser. No. 62/196,483 filed on Jul. 24, 2015.

### FIELD OF THE INVENTION

The present invention relates generally to body binders. More specifically, the present invention is abdominal and thoracic binder.

### BACKGROUND OF THE INVENTION

Individuals that suffer from permanent or long term hernia injuries wear binders or the like to prevent further injury or possible death. An abdominal binder is a fitted elastic material that wraps around the wearer's abdomen. Binders are typically used to support the wearer's muscles, as well as to keep bandages in place, and may help incisions heal after surgery. The existing binders that are currently available on the market have a number of notable disadvantages. First and foremost, existing binders lack comfort. The majority of binders currently available are not pleasant to wear for an extended period of time and needs frequent adjustments due to slippage. Additionally, binders that do not stay in place risks further injury as it is not performing its intended task. Therefore, it is the main objective of the present invention to provide an abdominal and thoracic binder that is comfortable to wear for extended periods of time without slippage. The present invention will be constructed from various different materials, each with their own purpose as well as added components such as adjustable stays for increased comfort and customizability.

Further, the existing binders are not very durable or breathable. Existing products lock in perspiration and absorbs water, causing discomfort and in some cases, leading to rashes. Existing binders lose their effectiveness when washed and wears quickly. Individuals that use abdominal binders on a daily basis must make multiple purchases a year, wasting money on the same product over and over again. Finally, current binders are not aesthetically pleasing and are not made to support exercise, swimming or active lifestyles in general. Therefore, it is another objective of the present invention to provide a binder that is durable, breathable, aesthetically pleasing and versatile. The present invention will feature components such as an adjustable closure to add to its durability, will have holes to increase breathability and will be able to be used in various settings such as the pool, the workplace, etc.

### BRIEF DESCRIPTION OF THE DRAWINGS

FIG. 1 is a front perspective view of the present invention in a flat configuration.

FIG. 2 is a rear perspective view of the present invention in the flat configuration.

FIG. 3 is a front view of the present invention in the flat configuration.

FIG. 4 is a rear view of the present invention in the flat configuration.

FIG. 5 is a magnified view of the present invention taken about a circle 5 in FIG. 4.

FIG. 6 is an exploded front perspective view of the present invention in the flat configuration.

FIG. 7 is a front perspective view of the present invention in a wrapped configuration.

FIG. 8 is a top view of the present invention in the wrapped configuration.

FIG. 9 is a schematic view of superimposed layers for the present invention without a padding layer.

FIG. 10 is a schematic view of superimposed layers for the present invention with the padding layer.

### DETAILED DESCRIPTION OF THE INVENTION

All illustrations of the drawings are for the purpose of describing selected versions of the present invention and are not intended to limit the scope of the present invention.

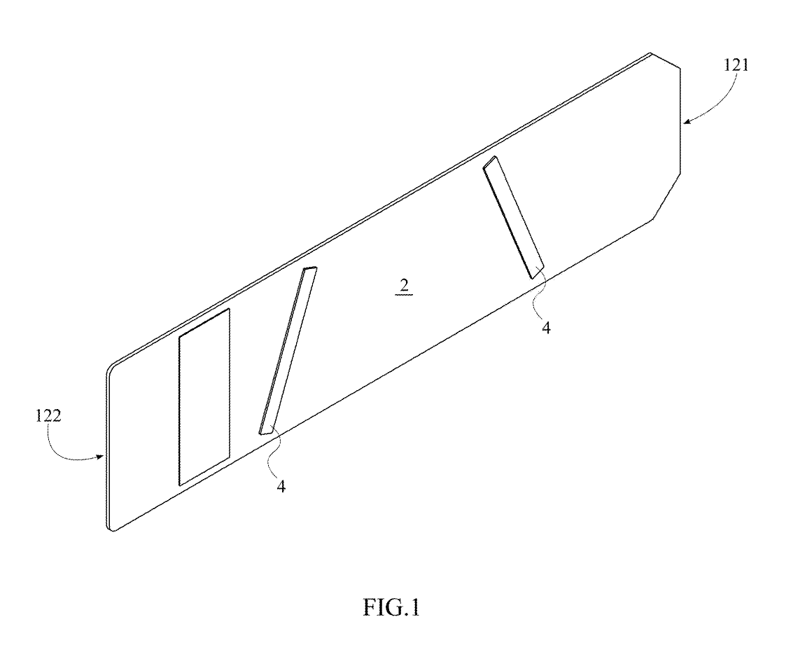

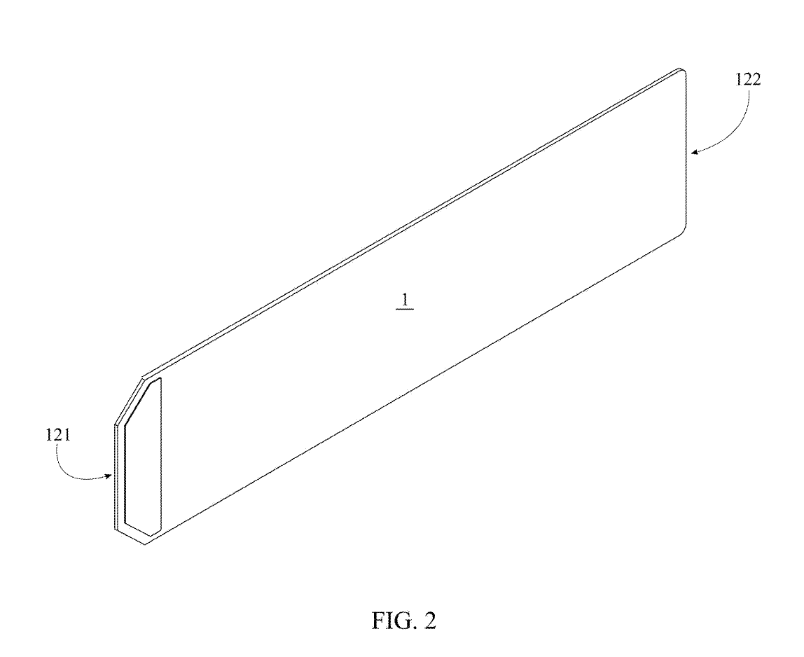

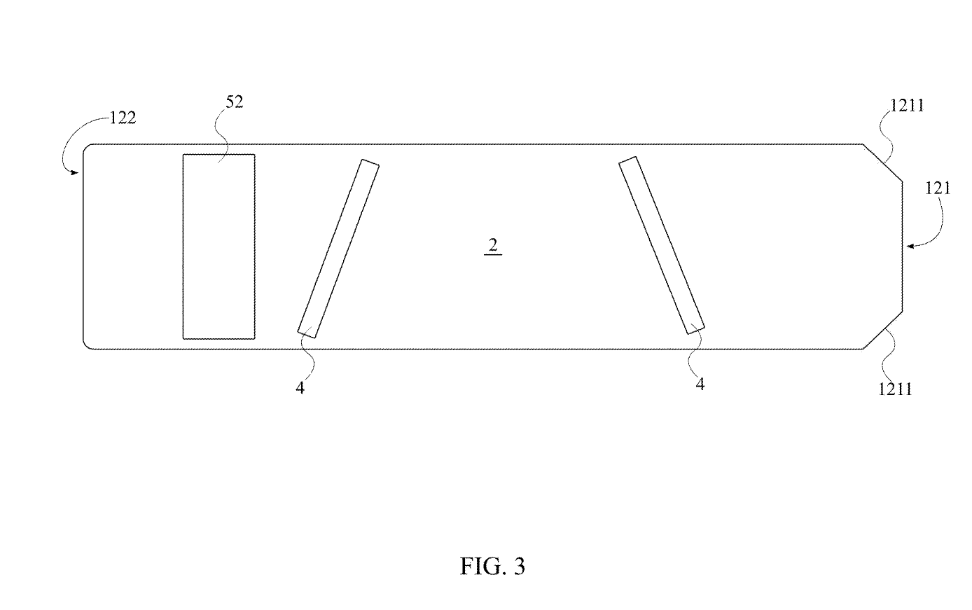

The present invention is an abdominal and thoracic binder that provides support to a user's abdomen and lower back muscles. The present invention is designed to be worn by a variety of users as the circumference of the present invention is adjustable. The present invention encourages ventilation and is designed to maintain its structure around the user's torso through any type of movement. The present invention comprises a porous band 1, a fastening layer 2, a plurality of ventilation holes 3, and a plurality of reinforcing rods 4. The porous band 1 and the fastening layer 2 each comprise a first end 121 and a second end 122. The porous band 1 comprises a fabric like material that directly applies slight pressure against the mid-section of the user. The porous band is shown in the rear perspective view of FIG. 2. The porous band 1 supports the engaged muscles of the user. The fastening layer 2 provides a mountable surface for the plurality of reinforcing rods 4. The plurality of ventilation holes 3 allows the skin covered by both the porous band 1 and fastening layer 2 to breath while surrounded by the porous band 1 and the fastening layer 2. The plurality of ventilation holes 3 prevents the user from sweating after prolonged wear of the apparatus. The plurality of reinforcing rods 4 supports the structure of the porous band 1 and the fastening layer 2 about the user's mid-section as illustrated in FIG. 1. The plurality of reinforcing rods 4 prevents the scrunching of the porous band 1 and the fastening layer 2 so that each of the plurality of ventilation holes 3 are not obstructed, as shown in FIG. 3.

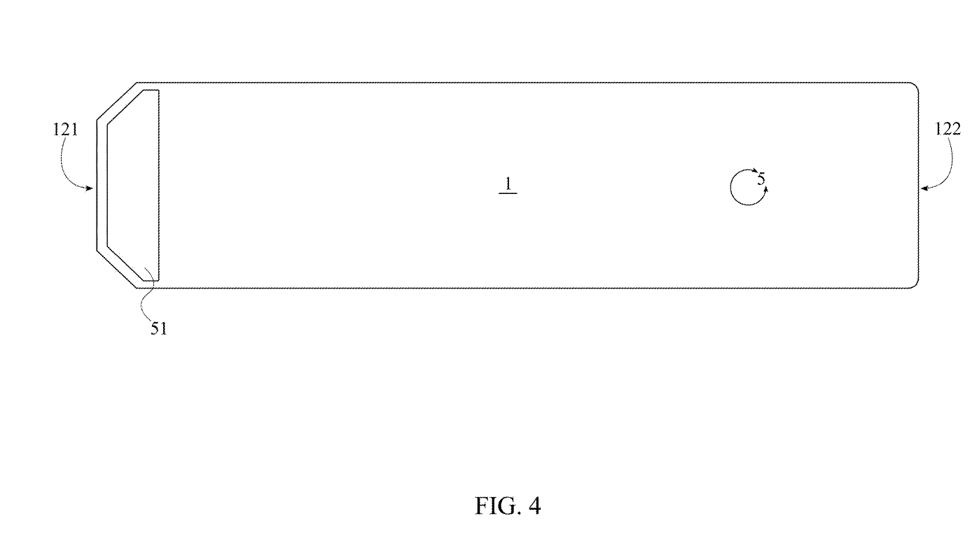

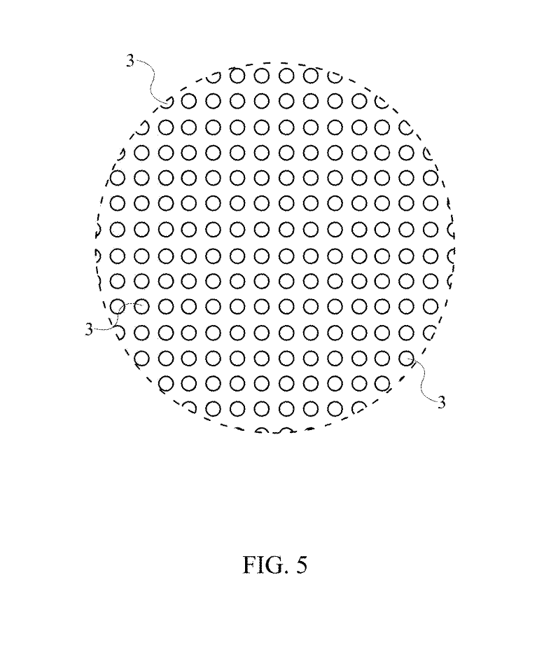

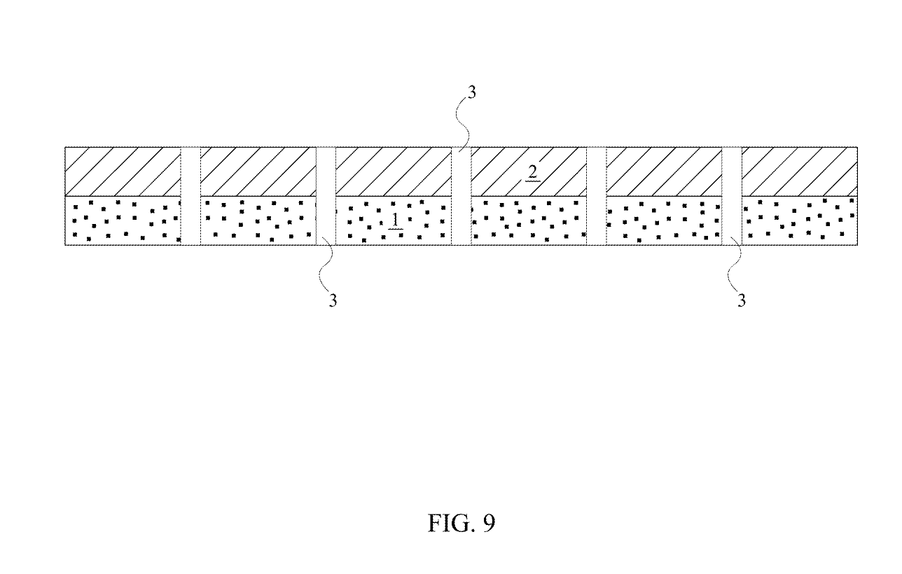

The general configuration of the aforementioned components allows the present invention to effectively support the mid-section of a user and encourage the breathability of the user's skin. The porous band 1 comfortably presses against the skin of the user. The fastening layer 2 is superimposed across the porous band 1 and is coextensive with the porous band 1. This arrangement between the fastening layer 2 and the porous band 1 prevents the fastening layer 2 from coming into contact with the user's skin. This coextensive arrangement is shown in the front view of FIG. 4. The plurality of ventilation holes 3 traverses through the porous band 1 and the fastening layer 2 and is distributed across the porous band 1 and the fastening layer 2, as shown in FIG. 9. The plurality of ventilation holes 3 allows air to be circulated from the user's skin, through the porous band 1, through the fastening layer 2, and into the area surrounding the present invention. The plurality of ventilation holes 3 is illustrated in FIG. 5. The plurality of reinforcing rods 4 is detachably attached to the fastening layer 2 and is preferably positioned adjacent the user's lower back. Consequently, the plurality of reinforcing rods 4 may be positioned in any desired arrangement according to the size and shape of the user's mid-section.

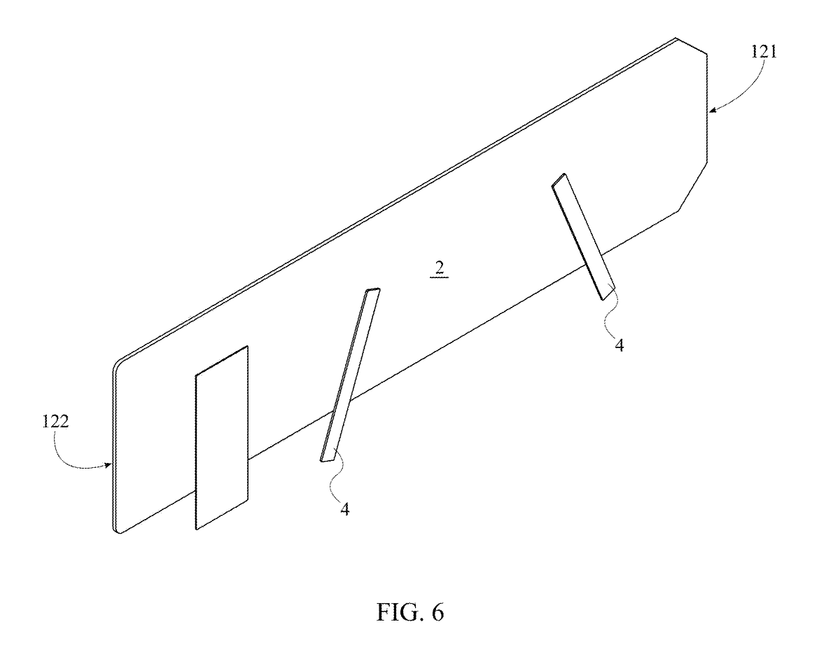

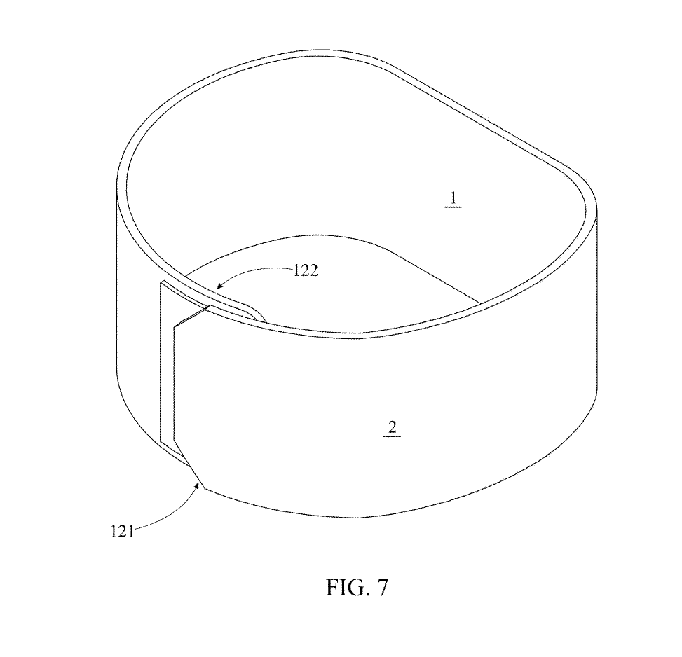

In order to effectively secure the present invention around the user's mid-section, the present invention further comprises a surface fastener 5, which secures the porous band 1 and the fastening layer 2 around the user's mid-section. The surface fastener 5 comprises a first interlocking member 51 and a second interlocking member 52, which are two separable pieces of the surface fastener 5 that can be readily attached together. Thus, the first interlocking member 51 is detachably attached to the second interlocking member 52. The engagement between the first interlocking member 51 and the second interlocking member 52 allows the user to adjust the circumference of the porous band 1 and fastening layer 2 about his or her mid-section. The preferred arrangement of the surface fastener 5 is such that the first interlocking member 51 is fixed onto the porous band 1 opposite the fastening layer 2, and the second interlocking member 52 is detachably attached to the fastening layer 2 opposite the porous band 1. This arrangement allows the ends of the present invention to overlap each other while the present invention encircles the user's midsection and allows the user to adjust the position of the second interlocking member 52 depending on the girth of the user's midsection. The second interlocking member 52 is shown as detached from the fastening layer 2 in FIG. 6. More specifically, the first interlocking member 51 is positioned adjacent the first end 121 of the porous layer 1, and the second interlocking member 52 is positioned adjacent the second end 122 of the fastening layer 2. The preferred arrangement of the surface fastener 5 allows the user to readily connect the first end 121 and the second end 122 of the porous band 1 and the fastening layer 2 respectively as shown in FIG. 7.

However, an alternate arrangement of the surface fastener 5 may comprise solely the first interlocking member 51. The first interlocking member 51 instead detachably attaches to the fastening layer 2. The first interlocking member 51 is fixed onto the porous band 1 opposite the fastening layer 2 and is positioned adjacent to the first end 121 of the porous layer 1. This alternate arrangement of the surface fastener 5 allows the first interlocking member 51 to directly engage with the fastening layer 2, decreasing the thickness of the overlap between the first end 121 and the second end 122 for both the porous band 1 and the fastening layer 2 respectively.

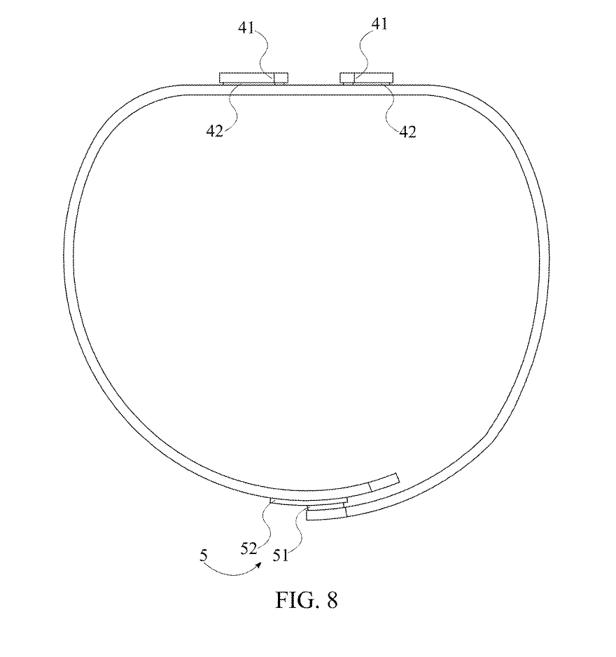

In order to properly secure the plurality of reinforcing rods 4 to the fastening layer 2, each of the plurality of reinforcing rods 4 further comprises a rod body 41 and a fastener strip 42. The rod body 41 and the fastener strip 42 of each of the plurality of reinforcing rods 4 are shown in FIG. 8. The fastener strip 42 is connected along the rod body 41. In addition, the rod body 41 is detachably attached to the fastening layer 2 by the fastener strip 42. The rod body 41 of each of the plurality of reinforcing rods 4 is rigid. The rod body 41 of each of the plurality of reinforcing rods 4 is engaged to define the structural shape of the porous body 1 and the fastening layer 2. The engagement of the plurality of reinforcing rods 4 with the fastening layer 2 prevents both the porous band 1 and fastening layer 2 from folding during the movements of the user.

In order to provide comfort while the present invention is worn by the user, the first end 121 of the porous band 1 further comprises a pair of corners 1211. The pair of corners 1211 is positioned opposite each other along the first end 121 of the porous band 1. More specifically, each of the pair of corners 1211 is a chamfered corner. The chamfered corners prevent a pointed end of the porous band 1 from poking the mid-section of the user. The first end 121 of the fastening layer 2 also comprises a pair of corners 1211, which are also positioned opposite each other along the first end 121 of the fastening layer 2. The pair of corners 1211 is chamfered corners, mirroring the pair of corners 1211 for the porous band 1. The chamfered corners of the fastening layer 2 further prevent the pointed end from poking the mid-section of the user.

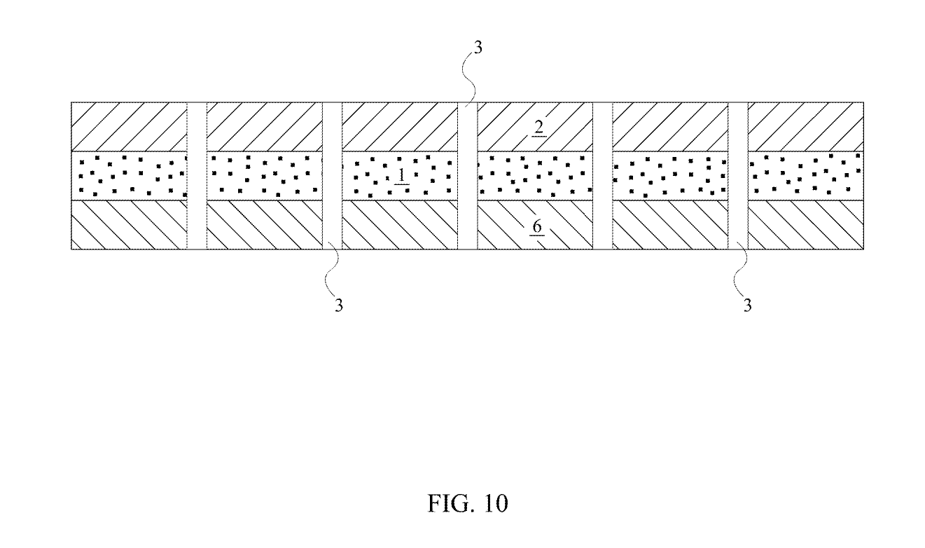

In some embodiments of the present invention, the present invention comprises a padding layer 6. The padding layer 6 provides additional cushioning against the user's mid-section and provides additional comfort to the skin directly in contact with the apparatus. Consequently, the padding layer 6 is superimposed across the porous band 1, opposite the fastening layer 2. This arrangement is illustrated in the schematic view of FIG. 10. The plurality of ventilation holes 3 further traverses through the padding layer 6. The plurality of ventilation holes 3 allows air to circulate to the padding layer 6 and therefore prevents unwanted sweating caused by the enclosure of the apparatus around the user's mid-section.

Although the invention has been explained in relation to its preferred embodiment, it is to be understood that many other possible modifications and variations can be made without departing from the spirit and scope of the invention as hereinafter claimed.

What is claimed is:

1. An abdominal and thoracic binder comprises:
a porous band;
a fastening layer;
a plurality of ventilation holes;
a plurality of reinforcing rods;
the porous band and the fastening layer each comprise a first end and a second end;
the fastening layer being superimposed across the porous band;
the fastening layer being coextensive with the porous band;
the plurality of ventilation holes traversing through the porous band and the adhering layer;
the plurality of ventilation holes being distributed across the porous band and the adhering layer; and
the plurality of reinforcing rods being detachably attached to the fastening layer.

2. The abdominal and thoracic binder as claimed in claim 1 comprises:
a surface fastener;
the surface fastener comprises a first interlocking member and a second interlocking member;
the first interlocking member being fixed onto the porous band opposite the fastening layer;
the second interlocking member being detachably attached to the fastening layer opposite the porous layer; and
the first interlocking member being detachably attached to the second interlocking member.

3. The abdominal and thoracic binder as claimed in claim 2 comprises:
the first interlocking member being positioned adjacent the first end of the porous layer; and
the second interlocking member being adjacent the second end of the adhering layer.

4. The abdominal and thoracic binder as claimed in claim 1 comprises:
a surface fastener;
the surface fastener comprises a first interlocking member;
the first interlocking member being fixed onto the porous band opposite the fastening layer; and
the first interlocking member being detachably attached to the fastening layer.

5. The abdominal and thoracic binder as claimed in claim 4, wherein the first interlocking member is positioned adjacent the first end of the porous layer.

6. The abdominal and thoracic binder as claimed in claim 1 comprises:
each of the plurality of reinforcing rods comprises a rod body and a fastener strip;
the fastener strip being connected along the rod body; and
the rod body being detachably attached to the fastening layer by the fastener strip.

7. The abdominal and thoracic binder as claimed in claim 1 comprises:
the first end of the porous band comprises a pair of corners;
the pair of corners being positioned opposite to each other along the first end of the porous band; and
the pair of corners being chamfered corners.

8. The abdominal and thoracic binder as claimed in claim 1 comprises:
the first end of the fastening layer comprises a pair of corners;
the pair of corners being positioned opposite to each other along the first end of the fastening layer; and
the pair of corners being chamfered corners.

9. The abdominal and thoracic binder as claimed in claim 1 comprises:
a padding layer;
the padding layer being superimposed across the porous band, opposite to the fastening layer; and
the plurality of ventilation holes further traversing through the padding layer.

10. An abdominal and thoracic binder comprises:
a porous band;
a fastening layer;
a plurality of ventilation holes;
a plurality of reinforcing rods;
a surface fastener;
a padding layer;
the porous band and the fastening layer each comprise a first end and a second end;
the fastening layer being superimposed across the porous band;
the fastening layer being coextensive with the porous band;
the plurality of ventilation holes traversing through the porous band and the adhering layer;
the plurality of ventilation holes being distributed across the porous band and the adhering layer;
the plurality of reinforcing rods being detachably attached to the fastening layer;
the surface fastener comprises a first interlocking member and a second interlocking member;
the first interlocking member being fixed onto the porous band opposite the fastening layer;
the second interlocking member being detachably attached to the adhering layer opposite the porous layer;
the first interlocking member being detachably attached to the second interlocking member;
the padding layer being superimposed across the porous band, opposite to the fastening layer; and
the plurality of ventilation holes further traversing through the padding layer.

11. The abdominal and thoracic binder as claimed in claim 10 comprises:
the first interlocking member being positioned adjacent the first end of the porous layer; and
the second interlocking member being adjacent the second end of the adhering layer.

12. The abdominal and thoracic binder as claimed in claim 10 comprises:
a surface fastener;
the surface fastener comprises a first interlocking member;
the first interlocking member being fixed onto the porous band opposite the fastening layer; and
the first interlocking member being detachably attached to the fastening layer.

13. The abdominal and thoracic binder as claimed in claim 12, wherein the first interlocking member is positioned adjacent the first end of the porous layer.

14. The abdominal and thoracic binder as claimed in claim 10 comprises:
each of the plurality of reinforcing rods comprises a rod body and a fastener strip;
the fastener strip being connected along the rod body; and
the rod body being detachably attached to the fastening layer by the fastener strip.

15. The abdominal and thoracic binder as claimed in claim 10 comprises:
the first end of the porous band comprises a pair of corners;
the pair of corners being positioned opposite to each other along the first end of the porous band; and
the pair of corners being chamfered corners.

16. The abdominal and thoracic binder as claimed in claim 10 comprises:
the first end of the fastening layer comprises a pair of corners;
the pair of corners being positioned opposite to each other along the first end of the fastening layer; and
the pair of corners being chamfered corners.
